(19) United States
(12) Patent Application Publication
(10) Pub. No.: US 2007/0258886 A1
(43) Pub. Date: Nov. 8, 2007

(54) Methods for assessing cell labeling

(75) Inventors: Eric T. Ahrens, Pittsburgh, PA (US); Paul Kornblith, Allison Park, PA (US)

Correspondence Address: ROPES & GRAY LLP;PATENT DOCKETING 39/41, ONE INTERNATIONAL PLACE, BOSTON, MA, 02110-2624, US

(73) Assignee: Celsense Inc., Pittsburgh, PA (US)

(21) Appl. No.: 11/787,520

(22) Filed: Apr. 16, 2007

Related U.S. Application Data
(60) Provisional application No. 60/792,242, filed on Apr. 14, 2006.

(51) Int. Cl. A61K 51/00; A61K 49/10; A61K 49/00

(52) U.S. Cl. 424/1.11; 424/9.34; 424/9.6

(57) ABSTRACT

The disclosure relates to methods for labeling cells ex vivo with agents that can be detected by MRI or PET techniques, or other techniques that permit non-invasive imaging of living organisms. The labeled cells can be re-administered to the patient and the movements of the labeled cells can be tracked in vivo by MRI, PET or other techniques. In part, the disclosed methods involve labeling a series of cell samples ex vivo determining the association of label with the cells, such that an appropriate dosage of labeled cells can be determined for each patient.

### RELATED APPLICATIONS

This application claims the benefit of U.S. Provisional Patent Application Ser. No. 60/792,242, filed on Apr. 14, 2006, the entire disclosure of which is incorporated herein by this reference.

### BACKGROUND

Many biological processes are carried out by dynamic, mobile populations of cells. For example, cells of the immune system are recruited from the bloodstream to areas of inflammation or infection, resulting in an accumulation of immune cells at the affected site. A marked infiltration of immune cells often occurs in tissues affected by autoimmune diseases, cancers and infections. Likewise, transplant rejection is mediated by host immune cells that enter and destroy the transplanted tissue. There is also growing evidence that stem cells originating in the bone marrow migrate through the bloodstream and assist in the regeneration of damaged tissues.

Typically, cell movements are monitored only in “snap shots” obtained by histological analysis of tissue biopsies. However, the process of sampling a tissue often alters the behavior of cells, and only a limited number of biopsies can be obtained from a particular tissue or organ.

Therefore, researchers have developed a variety of approaches for monitoring cells in vivo. Such approaches include detection of labeled cells by techniques that detect, e.g., light emission (fluorescence, luminescence), positron emission, gamma ray emission, emission of other particles by radioactive materials, and by nuclear magnetic resonance techniques (e.g., magnetic resonance imaging). Techniques based on nuclear magnetic resonance, positron emissions and radioactivity are generally more useful than light-based imaging technologies, as these latter are relatively ineffective at visualizing deep structures in optically opaque organisms.

In therapies based on the administration of cells or tissues (including, for example, bone marrow transplants and solid organ transplants) it will often be desirable to label the cells ex vivo to permit imaging in the patient. It may also be useful in a diagnostic context to administer a pool of labeled cells to a patient and subsequently track the location of such cells in the patient. Labeling may be achieved by administering label directly to an organism and hoping that a certain amount of label will become associated with cells of interest. Alternatively, cells of interest may be labeled ex vivo and then administered to the patient. Among the advantages of ex vivo labeling versus in vivo labeling, the ex vivo approach facilitates specific labeling of select cell populations, particularly populations of cells that will be administered for therapeutic or diagnostic purposes. Examples of ex vivo labeling systems are described, for example, in WO 2005/072780.

The disclosure provides methodologies that improve the safety and reliability of ex vivo cell labeling and the administration of labeled cells to patients.

### SUMMARY

In part, the disclosure provides a description of a previously unappreciated technical problem: the difficulty of obtaining relatively uniform dosages of in vivo imaging reagents where such agents are used to label cells ex vivo, followed by administration of the labeled cells to a patient. Animals that are commonly used for experimentation are bred for genetic homogeneity and raised according to standard protocols. Therefore it is often reasonable to suppose that a single protocol for labeling of cells ex vivo will yield consistent results in cells from different individuals of the same type of laboratory animal. By contrast, actual patients, whether human or non-human animals, are genetically heterogeneous and have diverse physiological states and complicating factors. Therefore a single protocol may not provide uniform labeling of cells from different patients. In part, methods disclosed herein may be used to adjust labeling conditions so as to obtain dosages of labeled cells that comply with safety parameters and are detectable in vivo.

In certain aspects, the disclosure provides methods and compositions for use in labeling cells ex vivo with an in vivo imaging reagent. In certain embodiments, the disclosure provides methods for assessing the ex vivo labeling of cells with an in vivo imaging reagent, where such cells are intended for administration to a patient. Labeled cells may be administered to a subject and subsequently detected by an in vivo (i.e., non-invasive) imaging technique. Examples of in vivo imaging techniques include positron emission tomography (PET) techniques, gamma cameras, SPECT (single-photon emission computed tomography), or nuclear magnetic resonance (NMR) techniques. Examples of NMR techniques include magnetic resonance imaging (MRI) and localized magnetic resonance spectroscopy (MRS). Because in vivo imaging techniques are generally performed as non-invasive procedures, the labeled cells may be detected at one or more time points in a living subject. Labeled cells may also be detected in a cell culture or in essentially any other milieu on which a nuclear magnetic resonance technique can be performed, such as tissue explants, organs and tissues removed from a subject (possibly prior to transplant into a transplant recipient), artificially generated tissues and various matrices and structures seeded with cells.

In certain aspects, the disclosure provides methods for assessing the labeling of cells intended for administration to a patient. A method may comprise contacting cells of a plurality of substantially identical cell samples with an in vivo imaging reagent; detecting the in vivo imaging reagent in the samples; and assessing the labeling of the cells with the in vivo imaging reagent. In another aspect, a method may comprise contacting cells of a plurality of substantially identical cell samples with an in vivo imaging reagent and a proxy reagent; detecting the proxy reagent in the samples; and assessing the labeling of the cells with the proxy reagent, thereby assessing the labeling of the cells with the in vivo imaging reagent. A proxy reagent will generally be an agent that is expected to label cells in a manner that is similar to that of the in vivo imaging reagent. The proxy reagent may itself be an in vivo imaging reagent, but preferred proxy reagents will be detectable by means that are less expensive, less time consuming and/or more widely available than the means for detecting the in vivo imaging reagent. For example, fluorescence (or other light emission) detection devices are widely available and generally require less capital investment than devices for magnetic resonance imaging or PET scanning, and therefore, fluorescent agents are a preferable category of proxy reagent. A proxy reagent may be, for example, selected from the group consisting of: a fluorescent or luminescent protein, a fluorescent or luminescent analogue agent, a fluorescent or luminescent dye, a colorimetric agent, or a radioactive agent. The in vivo imaging reagent may be stably associated with the proxy reagent to form a dual imaging reagent. In preferred dual imaging reagents, the in vivo imaging reagent is covalently linked to the proxy reagent. The term “substantially identical” as used in reference to cell samples is intended to indicate that the cell types that are present in each of the cell samples are similar enough that one of ordinary skill in the art would not ascribe significant differences in labeling of cells of the different samples to differences in the cells of each sample. Methods disclosed herein may also be used to compare cell samples that are not substantially identical. In methods of this type, it will often be useful to use substantially identical labeling conditions, while the cellular component of the samples differs. The cellular component may differ because the cells are of frankly different types (e.g., hepatocytes and cardiomyocytes) or because the cells in each sample, while derived from a common pool of cells, were cultured under differing conditions, or were derived from precursor or stem cells under varying differentiation protocols. The term “assessing” may mean any quantitative or, preferably, quantitative observation regarding the subject matter that is assessed. In a preferred embodiment, the assessment of labeling of cells comprises determining the average amount of label per cell, or another statistical representation of the degree of cellular labeling achieved (e.g., mean, median, standard deviation, or lower and upper ranges of labeling). Assessment of labeling of cells will preferably be performed so as to obtain information that will permit the selection of appropriate labeling conditions and/or an appropriate dose of labeled cells for administration to the subject.

Each of the substantially identical cell samples may be maintained in substantially identical conditions. In this instance, the methods disclosed herein are useful in evaluating the sample-to-sample reproducibility of the labeling methodology. Two or more of the substantially identical cell samples may be subjected to conditions that differ from each other (and usually such conditions will be pre-selected by the experimenter, although randomized or accidental testing of conditions are also contemplated), and in this instance, a method may further comprise assessing the effects of the differing conditions on the labeling of the cells with the in vivo imaging reagent. Often, it will be desirable to test a range of different conditions in a large number of samples, preferably at least 10, 20, 50, 100, 1000 samples, or more. Where large numbers of samples are to be tested, automated systems will be helpful, particularly automated, high throughput technology, such as automated plate loaders and plate readers, such as those adapted for 96-well or 384-well plates or plates with even greater numbers of wells. Thus, in a method disclosed herein, the cells may contacted with the in vivo imaging reagent (or contacted with the in vivo imaging reagent and the proxy reagent) by use of automated, high-throughput technology. Further, automated, high-throughput technology may be used to assess the labeling of the cells with the in vivo imaging reagent (including, where appropriate, the assessment of labeling with proxy reagent).

In vivo imaging reagents may be selected for use with any noninvasive imaging system. In certain preferred embodiments, the in vivo imaging reagent is an agent that is suitable for detection in vivo by a nuclear magnetic resonance technique.

Such an in vivo imaging reagent may be, for example, selected from the group consisting of: paramagnetic agents, superparamagnetic iron-oxide (SPIO) nanoparticles, Fe(3)O(4) nanoparticles, fluorocarbon imaging reagents, and Gd or Mn chelates (e.g., Gd-DTPA). An in vivo imaging reagent may be an agent that is suitable for detection by a non-invasive imaging technique selected from the group consisting of: positron emission tomography (PET), gamma ray detection, or SPECT (single-photon emission computed tomography).

Methods disclosed herein may be used to select safe, detectable dosages of labeled cells. For example, a method disclosed herein may further comprise determining conditions for labeling the cells with the in vivo imaging reagent that will allow the selection of a dosage of the cells for administration to a patient, wherein the dosage conforms to known safety parameters associated with the in vivo imaging reagent and provides adequate labeling to permit detection of the labeled cells in vivo. Similarly, method disclosed herein may further comprise administering to the patient a dosage of the cells, wherein the dosage conforms to known safety parameters associated with the in vivo imaging reagent and provides adequate labeling to permit detection of the labeled cells in vivo.

In certain aspects, a method further comprises testing cell samples with an additional assay. Additional assays are generally intended to assess biological characteristics that may have changed as a consequence of the labeling procedure. Biological characteristics that may be of interest include: cell number, cell viability, apoptosis, cell death, cell growth rate, entry into mitosis, expression of selected proteins and nucleic acids (e.g., proteins and nucleic acids that are indicative of a particular cell lineage, cell type or cell activation state), cell motility, chemoattraction, metabolic functions, and others. In certain embodiments, an additional assay may be selected from the group consisting of: a viability assay, a cell count, a cell cycle assay, a migration assay, and a functional assay.

Essentially any cell type that is desired may be used in the methods disclosed herein. Cells may be intended for administration to a patient, particularly a human patient, for a therapeutic purpose or a diagnostic purpose, or simply to allow detection of the localization of such cells in vivo. Cells may be autologous or allogeneic to the patient, as is appropriate to the clinical setting, however, it is expected that in most instances some effort will have been made to match certain genetic or protein expression characteristics. Examples of cells to be used include: blood cells, myoblasts, bone marrow cells, peripheral blood cells, umbilical cord blood cells, cardiomyocytes, chondrocytes, immune cells, fetal neural cells, neuronal precursors, fibroblasts, hepatocytes, islet cells of pancreas, keratinocytes and precursors of any of the preceding. Cells may be selected from the group consisting of: embryonic stem cells, cells cultured from embryonic stem cells, adult stem cells and cells cultured from adult stem cells. In some instances, the substantially identical cell samples are each a portion of a sample of cells obtained from the patient or a donor, or cultured from cells that were obtained from the patient or a donor. In some instances, the substantially identical cell samples are each from a common cell line or derived from a common cell line.

In certain aspects, the disclosure provides methods of administering a safe and useful dosage of labeled cells to a patient and detecting said labeled cells in vivo. Such a method may comprise:

a) contacting cells of a plurality of substantially identical cell samples with an in vivo imaging reagent;

b) detecting the in vivo imaging reagent in the samples;

c) assessing the labeling of the cells with the in vivo imaging reagent;

d) determining conditions for labeling the cells with the in vivo imaging reagent that will allow the preparation of a dosage of the cells for administration to a patient, wherein the dosage conforms to known safety parameters associated with the in vivo labeling agent and provides adequate labeling to permit detection of the labeled cells in vivo;

e) administering the dosage of the cells to the patient; and

f) detecting the labeled cells in vivo by a non-invasive imaging technique. Preferably, the non-invasive imaging technique in a nuclear magnetic resonance technique, particularly MRI.

In certain aspects, the disclosure provides methods for administering a safe and useful dosage of labeled cells to a patient and detecting said labeled cells in vivo, the method comprising:

a) contacting cells of a plurality of substantially identical cell samples with an in vivo imaging reagent and a proxy reagent;

b) detecting the proxy reagent in the samples;

c) assessing the labeling of the cells with the proxy reagent, thereby assessing the labeling of the cells with the in vivo imaging reagent;

d) determining conditions for labeling the cells with the in vivo imaging reagent that will allow the preparation of a dosage of the cells for administration to a patient, wherein the dosage conforms to known safety parameters associated with the in vivo labeling agent and provides adequate labeling to permit detection of the labeled cells in vivo;

e) administering the dosage of the cells to the patient; and

f) detecting the labeled cells in vivo by a non-invasive imaging technique. Preferably, the non-invasive imaging technique in a nuclear magnetic resonance technique, particularly MRI.

In certain aspects, the disclosure provides dual mode imaging reagents. Dual mode imaging reagents comprise an in vivo imaging reagent that is stably associated with a proxy reagent. The in vivo imaging reagent may be covalently or non-covalently bound to the proxy reagent. The proxy agent portion of the dual mode imaging reagent may be, for example, a dye or an expressed protein that has one or more of the following properties: fluorescent, luminescent, colored, fluorogenic, luminogenic and/or colorigenic.

Other aspects of the present disclosure will be apparent from the description below.

### DETAILED DESCRIPTION

1. Overview

This disclosure describes a process whereby one can rapidly and efficiently optimize the parameters for in vitro labeling of cells for in vivo detection. Here, “in vivo detection agent” will be understood to be an agent that is to be detected in a human being or other mammal by positron emission tomography (PET) techniques, gamma cameras, SPECT (single-photon emission computed tomography), or NMR techniques (e.g. MRI or MRS). “MRI label,” or “in vivo imaging reagent” will be understood to be any paramagnetic contrast agents or ¹⁹F tracer agents (e.g., perfluoropolyether emulsions) that can be detected using nuclear magnetic resonance techniques (e.g., MRI or MRS). “Imaging” and “detection” will be understood to be synonymous.

This process can be used, for example, in conjunction with a broad range of cellular therapeutics (e.g., immunotherapeutics, lymphocytes, stems cells, tissue transplants, etc.) and diagnostics where one uses in vivo magnetic resonance techniques to track the cells non-invasively in the human patient or animal after cell administration. This process can be used to standardize the cellular dose of the in vivo imaging reagent in vitro prior to the actual treatment to account for individual subject variability of cellular uptake properties of allogenic or autologous cells. Here, we refer to “cellular dose” or “cell dose” as the amount of in vivo imaging reagent that is contacted with the cell that in some way becomes associated with the cell's surface or otherwise taken up intracellularly. Alternatively, this process can be used to rapidly assess the cellular labeling parameters for a cell of an untried type. In some embodiments this process eliminates the need for expensive and complex nuclear magnetic resonance instrumentation (e.g., NMR, MRI, MRS) to measure the cellular dose. In preferred embodiments, this process uses high-throughput, parallel tissue culture preparations, proxy reagents, and rapid or automated cell-dose readouts using optical methods. Optionally, in addition to quantification of the cellular dose, in the same tissue culture preparation one can also assay other cellular properties, such as cell viability, phenotype, immunological function, in situ.

Effective development and practice of cellular therapeutics requires a means to non-invasively image or otherwise detect the cells post-transplantation. Certain classes of in vivo imaging reagents can tag or label cultured cells pre-administration, allowing their non-invasive visualization after they are delivered to the subject (i.e., human patient or animal) (WO2005072780, Granot et al., 2005, Magn Reson Med. October; 54(4):789-97; and Pintaske et al., 2005, Biomed Tech (Berl). June; 50(6):174-80). In order to ensure a consistent and safe dose (i.e., mass) of agent delivered to the cells before administration to a subject, one would like to be able to optimize the in vitro labeling or incubation conditions for the specific cell of interest, or the cells destined for a specific patient. For example, if a healthcare provider was planning to treat a patient with a malignancy using autologous lymphokine-activated lymphocytes, they could first contact the cultured lymphocytes with an in vivo imaging reagent, such as superparamagnetic iron-oxide (SPIO) nanoparticles (Pintaske et al., 2005, Biomed Tech (Berl). June; 50(6): 174-80) or a fluorochemical emulsion (WO2005072780) under physiological conditions before transfer to the patient. This contact would give an operable cell dose of the agent. Subsequently, this would allow the practitioner to non-invasively track the therapeutic cells after administration to the patient using non-invasive MRI.

When attempting to standardize the cell labeling process, one may encounter the problem that different sets of therapeutic or diagnostic cells, and particularly those that are obtained directly from a patient or donor or those that are cultured from cells obtained from a patient or donor, either allogenic or autologous, may have different responses to the labeling process. The uptake mechanism for the labeling agent may be, for example, endocytosis, macropinocytosis, transfection-assisted uptake, receptor-mediated endocytosis, etc., or any combination of these mechanisms. Thus one needs a way to rapidly calibrate the labeling incubation parameters (e.g., the agent concentration to add to the culture medium and/or the duration of incubation with the agent) to achieve consistent and safe doses of agents to the cells. Having a uniform, standardized cell dose for a particular type of therapeutic regimen is important for many reasons; some of these include: (i) minimizing the risk of overt toxicity to the cells due to over-labeling; (ii) minimizing the potential for under-labeling and a false negative MRI readout (i.e., an absence of signal or contrast in regions containing labeled cells in numbers such that they would normally show-up in the scan); and (iii) an essential first step in estimating or quantifying the number of labeled cells in a particular region of interesting in the body (e.g., at sites of metastases) on the basis of the MRI/MRS readout.

For example, in the case of a fluorochemical tracer cell label, the ability to consistently quantify the ¹⁹F signal changes is valuable clinically; these readouts can be used, for example, to estimate the delivery or trafficking efficiency of the cells to specific locations in the body. For example, a characteristic of intracellular fluorochemical tracer agent technologies (WO2005072780) is its potential to quantify the total ¹⁹F MRI/MRS signal in particular regions of interest containing labeled cells. The total ¹⁹F signal is directly related to the number of labeled cells in the region(s) showing signal.

Alternatively, if a paramagnetic contrast agent comprises the label, the ability to quantify contrast or nuclear magnetic resonance relaxation time (i.e., T1, T2 and/or T2*) associated changes in regions believed to contain the cells may reflect the concentration of cells in the region of interest, and this may have biological or clinical significance.

The ability to assess the number of therapeutic cells that have been implanted or that have migrated to the desired region, or otherwise, may be used to predict the efficacy of the treatment, and may, for example, direct additional follow-up treatments or procedures. In order to estimate or quantify the numbers of labeled cells in a region of interest, one should first calibrate, or in some way control for, the average amount of label dose per cell for a population that will ultimately be administered to the subject. Individual variability may be possible, for example, even when a cell type is used that was selected for a particular phenotype. This variability may be due to a variety of reasons (e.g., cellular health, genetic differences, previous treatment, etc.), and this in turn could affect the cell's ability to become labeled.

In addition to individualized labeling of similar cell types, the ability to rapidly assess the cell culturing parameters for labeling different cell types or untried cells types, is highly useful. Generally, different cells types that are contemplated for cellular therapeutics, or are otherwise of interest for in vivo trafficking studies utilizing MRI, will have different innate abilities to take up agents. For example, some cells are highly phagocytic and readily take up particulate labeling agents in culture, while other cell types will not take these agents up unless they are assisted, via transfection agents or peptides, for example. Regardless of the cellular uptake mechanism, it is useful to have a rapid or high-throughput approach for determining the set of tissue culture parameters to optimally label a particular cell type. In the context of this process, “optimally labeled” means that there is sufficient labeling agent associated with cells such that they can be detected with in vivo imaging techniques, but not so much that there is overt cytotoxicity or other deleterious effects to the cells or to the patient upon administration of the labeled cells. The initial starting point for label optimization of an untried cell type can be the parameters used for a previously tried cell type where the labeling has been characterized. For example, if one has previously determined the parameters for labeling lymphocytes, and now wants to determine the appropriate parameters for a different cell type such as a dendritic cell, it would be useful to have a rapid or high-throughput approach for determining the new set of optimal parameters using the lymphocyte's parameters as a starting point.

2. In Vivo Detection Techniques

As described herein, nuclear magnetic resonance techniques may be used to detect populations of labeled cells. The term “detect” is used to include any effort to ascertain the presence or absence of a labeled molecule or cell, particularly by a nuclear magnetic resonance technique. The term “detect” is also intended to include more sophisticated measurements, including quantitative measurements and two- or three-dimensional image generation. For example, MRI may be used to generate images of such cells. In many instances, the labeled cells may be administered to a living subject. Following administration of the cells, some portion of the subject, or the entire subject, may be examined by MRI to generate an MRI data set. A “data set”, as the term is used herein, is intended to include raw data gathered during magnetic resonance probing of the subject material, as well as information processed, transformed or extracted from the raw data. Examples of processed information include two-dimensional or three-dimensional pictorial representations of the subject material. Another example of extracted information is a score representing the amount or concentration of imaging reagent or ¹⁹F signal in the subject material. For example, the signal-to-noise-ratio (SNR) of the ¹⁹F signal may be measured and used to calculate the abundance of labeled cells. This type of data may be gathered at a single region of the subject, such as, for example, the spleen or another organ of particular relevance to the labeled cells. Labeled cells may be examined in contexts other than in the subject. It may be desirable to examine labeled cells in culture. In certain embodiments, labeled cells may be applied to or generated within a tissue sample or tissue culture, and labeled cells may therefore be imaged in those contexts as well. For example, an organ, tissue or other cellular material to be transplanted may be contacted with an imaging reagent to generate labeled cells prior to implantation of such transplant in a subject.

In general, labeling agents of the invention are designed for use in conventional MRI detection systems. In the most common implementation of MRI, one observes the hydrogen nucleus (proton, ¹H) in molecules of mobile water contained in subject materials. To detect labels disclosed herein, an alternate nucleus is often detected, ¹⁹F. ¹⁹F MRI has only slightly less intrinsic sensitivity compared to ¹H; the relative sensitivity is approximately 0.83. Both have a spin of +½. The natural isotopic abundance of ¹⁹F is 100%, which is comparable to 99.985% for ¹H. The physical principles behind the detection and image formation are the same for both ¹H and ¹⁹F MRI. The subject material is placed in a large static magnetic field. The field tends to align the magnetic moment associated with the ¹H or ¹⁹F nuclei along the field direction. The nuclei are perturbed from equilibrium by pulsed radio-frequency (RF) radiation at the Larmor frequency, which is a characteristic frequency proportional to the magnetic field strength where nuclei resonantly absorb energy. Upon removing the RF, the nuclei induce a transient voltage in a receiver antenna; this transient voltage constitutes the nuclear magnetic resonance (NMR) signal. Spatial information is encoded in both the frequency and/or phase of the NMR signal by selective application of magnetic field gradients that are superimposed onto the large static field. The transient voltages are generally digitized, and then these signals may be processed by, for example, using a computer to yield images.

At constant magnetic field strength, the Larmor frequency of ¹⁹F is only slightly lower (−6%) compared to ¹H. Thus, it is straightforward to adapt conventional MRI scanners, both hardware and software, to acquire ¹⁹F data. The ¹⁹F detection may be coupled with different types of magnetic resonance scans, such as MRI, MRS or other techniques. Typically, it will be desirable to obtain a ¹H MRI image to compare against the ¹⁹F image. In a living organism or other biological tissue, the proton MRI will provide an image of the subject material and allow one to define the anatomical context of the labeled cells detected in the ¹⁹F image. In a preferred embodiment of the invention, data is collected for both ¹⁹F and ¹H during the same session; the subject is not moved during these acquisitions to better ensure that the two data sets are in spatial registration. Normally, ¹⁹F and ¹H data sets are acquired sequentially, in either order. Alternatively, with appropriate modifications to the hardware and/or software of the MRI instrument, both data sets can be acquired simultaneously, for example, to conserve imaging time. Other imaging techniques, such as fluorescence detection or PET may be coupled with ¹⁹F MRI. This will be particularly desirable where a fluorocarbon imaging reagent has been derivatized with a fluorescent moiety, or in the case of PET, the agent incorporates both ¹⁸F and ¹⁹F isotopes.

MRI examination may be conducted according to any suitable methodology known in the art. Many different types of MRI pulse sequences, or the set of instructions used by the MRI apparatus to orchestrate data collection, and signal processing techniques (e.g. Fourier transform and projection reconstruction) have been developed over the years for collecting and processing image data (for example, see Magnetic Resonance Imaging, Third Edition, editors D. D. Stark and W. G. Bradley, Mosby, Inc., St. Louis Mo. 1999). The agents and methods of this invention are not tied to any particular imaging pulse sequence or processing method of the raw NMR signals. For example, MRI methods that can be applied to this invention broadly encompasses spin-echo, stimulated-echo, gradient-echo, free-induction decay based imaging, and any combination thereof. Fast imaging techniques, where more than one line ink-space or large segments of k-space are acquired from each excited signal, are also highly suitable to acquire the ¹⁹F (or ¹H) data. Examples of fast imaging techniques include fast spin-echo approaches (e.g. FSE, turbo SE, TSE, RARE, or HASTE), echo-planar imaging (EPI), combined gradient-echo and spin-echo techniques (e.g. GRASE), spiral imaging, and burst imaging. The development of new and improved pulse sequence and signal processing methods is a continuously evolving field, and persons skilled in the art can devise multiple ways to image the ¹⁹F labeled cells in their anatomical context.

As another example of a nuclear magnetic resonance technique, MRS can be used to detect the presence of fluorocarbon-labeled cells in localized tissues or organs. Normally MRS methods are implemented on a conventional MRI scanner.

3. In Vivo Imaging Reagents and Formulations

In some embodiments the in vivo imaging reagent is a in vivo imaging reagent that may include paramagnetic agents, superparamagnetic iron-oxide particles, magnetite particles, fluorocarbon (e.g., PFPEs), and Gd or Mn chelates. Magnetite, Fe₃O₄, is composed of an Fe(II) atom and two Fe(III) atoms.

In certain embodiments, the in vivo imaging reagent used in the subject methods is a fluorocarbon, i.e., a molecule including at least one carbon-fluorine bond. By virtue of the ¹⁹F atoms, the imaging reagents disclosed herein may be detected by ¹⁹F MRI and other nuclear magnetic resonance techniques, such as MRS techniques. In certain preferred embodiments, a fluorocarbon imaging reagent will have one or more of the following properties: 1) tolerable cytotoxicity; 2) a ¹⁹F NMR spectrum that is simple, ideally having a single, narrow resonance to minimize chemical shift artifacts; 3) high sensitivity with a large number of NMR-equivalent fluorine atoms in each molecule; 4) formulated to permit efficient labeling of many cell types and not restricted to phagocytic cells.

Exemplary compounds include aryl or heteroaryltrifluoromethyl sulfonic acid esters (triflates) or sulfonamides (triflamides), esters of fluorinated alcohols (such as 2,2,2-trifluoroethanol, perfluoro-tert-butanol, and 2,2,3,3,3-pentafluoropropanol), esters and amides of perfluoroalkanoic acids (such as trifluoroacetic acid, perfluorotetradecanoic acid, and nonafluoropentanoic acid), ethers of perfluoroalkanes, and the like. Preferably, the imaging reagent comprises a plurality of fluorines bound to carbon, e.g., greater than 5, greater than 10, greater than 15 or greater than 20 fluorines bound to carbon. Preferably, at least 4, at least 8, at least 12 or at least 16 of the fluorines have a roughly equivalent NMR chemical shift.

In certain embodiments, the imaging reagent is a perfluoro crown ether, such as perfluoro-15-crown-5, perfluoro-18-crown-6, perfluoro-12-crown-4, etc., also referred to herein as cyclic perfluoropolyethers (cyclic PFPEs). Such compounds are advantageous in that the ¹⁹F nuclei of these molecules will have similar or identical NMR resonances, resulting in a higher signal-to-noise ratio image with a reduction in or absence of chemical-shift image artifacts. The macrocycle perfluoro-15-crown-5 ether has particularly preferable characteristics. It is neither lipophilic nor hydrophilic, which is typical for perfluoropolyethers, and is emulsified into aqueous solution. Typical emulsions are small particulates (−10-500 nm diameter) that are stable in aqueous solution and can be taken up by cells. One of skill in the art will recognize that other fluorinated compounds will have desirable properties, particularly those fluorinate compounds in which each fluorine atom is in a similar chemical environment. Esters of perfluoro-tert-butanol, 1,3,5-tris(trifluoromethyl) benzene, hexafluoroacetone, poly(trifluoromethylethylene), andperfluorocyclohexane are examples of compounds having multiple fluorine atoms with ¹⁹F resonances that have the same, or nearly the same, Larmor frequencies.

In certain embodiments, the imaging reagent is a polymer. In certain embodiments, the imaging reagent is or includes a linear perfluoropolyether (linear PFPE), e.g., a compound having a structure or portion thereof comprising repeated units of-[O—CF2 (CF2) xCF2]n-, where x is an integer from 0 to 10, preferably from 0-3, and n is an integer from 2 to 100, preferably from 4 to 40. Perfluorinated linear polyethylene oxide, for example, can be obtained from Exfluor Corp. (Round Rock, Tex.). Either or both ends (or a plurality of ends, in the case of branched polymers) may be derivatized with a moiety that provides an additional desired functionality.

For example, an imaging reagent may have a formula of A-B—C, where A and/or C may be a functional moiety and B comprises repeated units of-[O—CF2 (CF2) xCF2], where x is an integer from 0 to 10, preferably from 0-3, and n is an integer from 2 to 100, preferably from 4 to 40. Functional moieties (e.g., non-fluorinated monomers conferring a particular desired function) are discussed further below.

A linear perfluoropolyether may also be described as a composition having the average formula:
XO(Y—O)nZ
wherein Y is selected from the group consisting of: 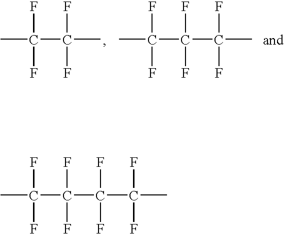
Wherein n is an integer from 8 to 30; wherein X and Z are the same and are selected from the group consisting of perfluoroalkyls, perfluoroethers, fluoroalkyls terminated with fluoroacyl, carboxyl, amide or ester, methylols, acid chlorides, amides, amidines, acrylates and esters, as well as any of the preceding derivatized with a functional moiety.

While a completely fluorinated polymer can be formed, for example, by reacting a perfluorinated diacid with a perfluorinated dihalocarbon (such as 1,4-diiodooctafluorobutane), fluorinated monomers can be reacted with other monomers (optionally functional moieties, which may be non-fluorinated) to form hybrid polymers that are useful as imaging reagents. A variety of different non-fluorinated monomers can be used to vary the chemical and physical properties of the overall polymer, and make it possible to tailor the imaging reagent for specific uses. For example, a highly lipophilic imaging reagent may concentrate in adipocytes and other fatty tissues, while a highly hydrophilic imaging reagent may be useful for imaging the circulatory system or the lymph system.

Another PFPE composition of interest is linear PFPEs derivatized with a variety of end groups. The linear compounds have the advantage that one can conjugate a variety of functional entities to the end groups, such as functional moieties of various types. The ¹⁹F NMR spectra of these linear compounds generally are more complex than the macrocyclic compounds, but a PFPE with two well-separated NMR signals can also be used. In this case it may be desirable to use an MRI pulse sequence that incorporates one or more off-resonance saturation pulses applied to the smaller resonance to eliminate any chemical shift artifacts.

In certain embodiments, a linear perfluoropolyether may be derivatized with a relatively hydrophilic moiety at one, or preferably, both ends. For example, the hydrophilic moiety may be a polyethylene glycol, thus forming a tri-block copolymer with water-soluble regions on each end and a hydrophobic region in the center. When mixed in an aqueous environment, imaging reagents of this type will tend to form micelles, with the PFPE core surrounded by a water-soluble coat. Amino-PEG blocks are commercially available with a range of molecular weights.

In certain embodiments, the invention provides formulations of imaging reagents that are suitable for uptake by cells. Emulsions comprising a fluorocarbon imaging reagent, such as a PFPE, will preferably have a distribution of particle sizes that allow adequate cellular uptake. For example, it will generally be desirable that the mean particle size fall within a range from 10 nm to 500 nm, and preferably a range of from 30 nm to 150 nm or a range of from about 350 to 500 nm. Optionally, 25%, 50%, 75% or more of the particles will also fall within the selected range.

Particle sizes may be evaluated by, for example, light scattering techniques or by visualizing the emulsion particles using EM micrographs. In certain cell types that have a relatively small amount of cytoplasm, such as most stem cells, preferred particle sizes will be in the range of 10-50 nm in diameter. Emulsions for use in cells should preferably be stable at a wide range of temperatures. For example, it will often be desirable to store the emulsion at a cool temperature, in the range of 2-10 C, and preferably 4 C, and then warm the emulsion to room temperature (e.g., 18 to 28 C, and more typically 20 to 25 C). After labeling of cells, the emulsion will experience a temperature of about 37 C. Accordingly, a preferred emulsion will retain the desired range of particle sizes at temperatures ranging from refrigeration temperatures up to body temperature. The surfactant may be designed to form stable emulsions that carry a large quantity of PFPE into the aqueous phase. Additionally, it may have properties that increase the intracellular delivery of the emulsion particles in the shortest possible incubation time. Increasing the PFPE intracellular loading improves sensitivity to the labeled cells. Furthermore, decreasing the incubation time can be important when working with the primary cells cultures because the cell phenotype may evolve over time. The efficiency of intracellular uptake depends on cell type. For example macrophages and dendritic cells will endocytose almost any particulate, whereas other cell types of interest may only be weakly phagocytic. In either case the uptake efficiency can be boosted substantially by incorporating cationic lipids into the surfactant, by using peptides (e.g. oligo-Arg9 and TAT-like peptides), or by incorporating antibodies that target specific cell surface molecules.

The properties of an emulsion may be controlled primarily by the properties of the imaging reagent itself, the nature of surfactants and/or solvents used, and the processing (e.g., sonication, etc.). Methods for forming PFPE emulsions are extensively described in U.S. Pat. Nos. 5,330,681 and 4,990,283. A continuous phase of a polyhydroxylated compound, such as polyalcohols and saccharides in concentrated aqueous solution may be effective. The following polyalcohols and saccharides have proved to be particularly effective: glycerol, xylitol, mannitol, sorbitol, glucose, fructose, saccharose, maltitol, dimer compounds of glycerol (di-glycerol or bis(2,3-di-hydroxypropyl)ether, solid water soluble polyhydroxylated compounds as sugars and glycerol condensation products as glycerol and tetraglycerol. The dispersion in emulsion may be performed in the presence of conventional surfactants, including cationic, anionic, amphoteric and non-ionic surfactants, with ionic surfactants being preferable. Examples of suitable surfactants include sodium lauryl sulphate, sulphosuccinate (sulphosuccinic hemiester), coco-amphocarboxyglycinate, potassium cetyl phosphate, sodium alkyl-polyoxyethylene-ether carboxylate, potassium benzalconium chloride, alkyl amidopropyl betaine, cetyl-stearilic ethoxylated alcohol, and sorbitan-ethoxylate (20)-mono-oleate Tween 20. While thermodynamic equations may be used to attempt to predict mixtures of imaging reagents that will give emulsions having the desired particle sizes and stability, it is generally accepted that actual testing of various mixtures will be most effective. The emulsification of mixtures is simple and quick, permitting rapid testing of a wide range of combinations to identify those that give rise to emulsions that are suitable for use in the methods disclosed herein.

In certain embodiments the in vivo imaging reagent is an agent suitable for PET techniques, gamma cameras, or SPECT.

4. Proxy Reagents

Imaging reagents that permit fluorescent detection are particularly useful in a variety of applications. For example, fluorescent labeling permits the use of fluorescence-based cell sorting mechanisms, such as Fluorescence Activated Cell Sorting (FACS). Cell sorting may be desirable, for example, to enrich for a population of cells that have been successfully labeled. This may be particularly useful where labeling has been directed to rarer cell populations. Proxy reagents may be dual mode agents, which are additionally useful for finding and characterizing labeled cells after they have been implanted into a living subject. In this application, cells may be biopsied, or by some other means harvested, from the subject after they have resided there for some duration. Biological analysis of the harvested cells can then be performed. For example, FACS analysis can be performed on the harvested cells, where after positively selecting cells for the fluorescent PFPE label, the cells can be assayed for the expression of specific cell surface markers (using a different color fluorescent probe) to investigate any change in cell phenotype that occurred following implantation. Fluorescent labels may also be used for fluorescence microscopy of cells, particularly using three-dimensional confocal fluorescence microscopy.

In some embodiments a proxy reagent is a fluorescent analogue agent made of the in vivo imaging reagent. This agent preferably has similar or nearly-identical chemical and physical properties (e.g., effective electronic charge, molecular weight, steric size, confirmation, etc.) as the actual in vivo imaging reagent except that it exhibits fluorescent properties that are readily detectable. Alternatively one could use an agent that is “dual-mode” in that it is detectable in vivo and also contains a fluorescent moiety. For example, in the case of PFPE nanoparticle agents, the fluorescent moieties may be conjugated to the PFPE or surfactant molecules before emulsification (see WO2005072780, herein incorporated in its entirety by reference). Examples of fluorescent moieties include: fluorescein, lissamine, phycoerythrin, rhodamine (Perkin Elmer Cetus), Cy2, Cy3, Cy3.5, Cy5, Cy5.5, Cy7, FluorX (Amersham) and Alexa dyes (Molecular Probes). Fluorescent moieties include derivatives of fluorescein, benzoxadioazole, coumarin, eosin, Lucifer Yellow, pyridyloxazole and rhodamine. These and many other exemplary fluorescent moieties may be found in the Handbook of Fluorescent Probes and Research Chemicals (2000, Molecular Probes, Inc.). Additional fluorescent moieties include fluorescent nanocrystals, such as the “quantum dot” products available from Quantum Dot Corporation (Hayward, Calif.). Such nanocrystals may be constructed with a semiconductor core having an appropriate emission spectrum (e.g., CdS, CdSe, CdTe), a shell composed of a non-emissive transparent and relatively non-reactive material that can be efficiently wed to the underlying core material (e.g., ZnS), and a coating that provides desirable solubility (e.g., for solubility in aqueous, physiological solutions) and possible reactive groups for attachment to a fluorocarbon described herein. Alternatively the fluorescent moiety could be a dye (e.g., lipophilic dye such as DiI or DiO) that binds tightly to the surfactant on the surface of the PFPE emulsion particle.

Detection moieties suitable for PET imaging may also be used to create dual mode imaging reagents that are detectable by nuclear magnetic resonance techniques and by PET techniques. For example, the ¹⁸F isotope is a potent label for PET detection methods. A fluorocarbon imaging reagent may comprise a mixture of ¹⁸F and ¹⁹F isotopes, thus providing a dual mode label that is suitable for MRI/MRS and PET. ¹⁸F and ¹⁹F may also be added in separate monomers to form a mixed copolymer, or ¹⁸F portions may be located at either end of a linear polyether, at the position where most other functional moieties would be added. ¹⁸F has no NMR signal and so may be added at positions that would, for example, tend to decrease NMR line width, simplify the NMR spectrum, or alleviate chemical shifts from resonances that adversely effect the read-out obtained by a nuclear magnetic resonance technique. In addition, molecules of the fluorocarbon imaging reagents can incorporate other radioisotopes that are effective PET probes, such as ¹¹C, 15O, and ¹³N. Those skilled in the art can, in view of this specification, devise many other PET-detectable moieties that can be incorporated into or, for example, attached to an end group (s), of the imaging reagents of this invention.

In the case of paramagnetic agents, several types of fluorescent analogues are known and described in the literature (Venanzi et al., 2004, Biopolymers. October 5; 75(2): 128-39). For example, certain lanthamides such as europium and terbium are know to have fluorescent properties, and analogue in vivo imaging reagents can be synthesized by substituting these metal ions for the usual MRI-active gadolinium (Gd) as part of a chelated complex (Elster et al., 1989, AJNR Am J Neuroradiol. November-December;10(6):1137-44) for Eu-DTPA, Eu-DOTA, Tb-DTPA, Tb-DOTA, etc.). Dual-mode paramagnetic agents can also be synthesized, for example, by complexing rhodamine, fluorescein, or cyanine moieties with Gd- or Fe-based MRI/MRS agents (Schneider et al., 2000, Invest Radiol. September; 35(9):564-70). In preferred embodiments, before the read-out, the excess label that does not become associated with the cells (i.e. that which is left in suspension) is washed away via standard methods and the cells are re-immersed in fresh medium or buffer; this eliminates the “background” fluorescence signals from the wells.

In some embodiments luminescent dyes [e.g. Tris(2,2′-bipyridyl) dichlororuthenium (II) hexahydrate and platinum octaethylporphyrin ketone] comprise proxy reagents in any of the ways described above. In addition such dyes may be doped inside a silica network such as polydimethysiloxane for cellular delivery.

Alternatively, the in vivo imaging reagent can be contacted, attached, or in some way associated with a nucleic acid sequence coding for an optically-visible reporter. Exemplary optical reporters include, but are not limited to, green fluorescent protein (GFP) and related derivatives (YFP, BFP, etc.) or a variety of luciferases. Plasmids, or any other nucleic acid shuttle, containing these reporters can be in some way bound to the in vivo imaging reagent. For example, plasmids containing optical reporters can be incorporated into a transfection agent that also coats the imaging reagent. As a further example, the nucleic acid shuttle can be made, by a variety of means, adherent to the surfactant covering the PFPE nanoparticle's surface. Oligonucleotides may also be directly conjugated to nanoparticles (Glynou et al. 2003, Anal Chem. August 15; 75 (16):4155-60) and proteins as well (Abrahamsson et al., 2004, Biosens Bioelectron. June 15; 19 (11):1549-57). Direct DNA or protein tags could allow further association with a reporter by a targeting moiety, hybridization or other means known to one skilled in the art.

Upon incubation of the cells at physiological temperatures, the cells will produce the reporter gene product. Subsequently, the amount of light emitted by the reporter per culture well at the appropriate wavelength will be proportional to the number of labeling agent particles taken up by the cells per well during the incubation period. In this way one has a direct measure of the mean labeling agent incorporated by the cells per well. An advantage of using the genetically-encoded reporters is that there is no need to use a wash step before the read-out of the uptake; extracellular nucleic acid reporter that has not been incorporated intracellularly will be optically quiescent, i.e., not produce light that can be detected at the wavelength appropriate for the reporter gene product. Secondarily, by using genetically-encoded reporters there is no need to develop or manufacture fluorescent analogues or dual-mode agents that are similar to the actual in vivo imaging reagent used in the patient or subject.

Alternatively, one can construct proxy reagents that modulate the optical absorbance or color spectrum when associated with labeled cells. These agents will be understood to be colorimetric agents. For example, one can use the nucleic acid-based reporter gene coding for beta-galactosidase (β-Gal). In a similar fashion as described for the nucleic acid fluorescent probes (above), plasmids, or any other gene carrier containing β-Gal can be bound in some way to the MRI/MRS labeling agent. Upon incubation of the labeling agent and cells at physiological temperatures, the cells will produce the exogenous enzyme β-Gal after the labeling agent complex is taken up. Cells in each well can then be stained using standard chemical methods (i.e., X-Gal blue staining); the degree of cellular labeling per well is assayed via detection of its colorimetric change. Similarly, other enzymes such as horse-radish peroxidase (HRP) or moieties that are recognized by commercially available antibodies such as digoxigenin can be bound to the labeling agent.

In certain embodiments a radioactive proxy reagent is used. For example, by making in vivo imaging reagent analogues that incorporate radioactive ⁵⁷Fe or ⁵⁹Fe for SPIO agents, or ¹⁸F for the PFPE tracer agent types. After the incubation period, and a wash step to remove excess (i.e., non-associated) agent, the number of radioactive decay events per unit time per well is proportional to the amount of in vivo imaging reagent taken up by the cell. The radioactive label may also be a dual mode agent or be indirectly associated with the in vivo imaging reagent.

5. Cell Types

Methods described herein may be used with a wide range of cells, including both prokaryotic and eukaryotic cells, and preferably mammalian cells. Technologies for cell preparation include cell culture, cloning, nuclear transfer, genetic modification and encapsulation. Cells will preferably be those that are obtained from a donor and intended for administration to a patient. The patient may, in many instances, be the donor. Cells may be primary or secondary cultures of those obtained from a donor, but generally cells referred to herein as “cultured from cells obtained from a donor” are not cells that have been used to generate a cell line or cells that have been cultured repeatedly to give rise to cells that are substantially different from those obtained from the donor. These latter cell types are encompassed within the term “cell line”.

A partial list of suitable mammalian cells includes: blood cells, myoblasts, bone marrow cells, peripheral blood cells, umbilical cord blood cells, cardiomyocytes (and precursors thereof), chondrocytes (cartilage cells), dendritic cells, fetal neural tissue, neuronal precursors, fibroblasts, hepatocytes (liver cells), islet cells of pancreas, keratinocytes (skin cells) and stem cells. In some embodiments the cells compose tissue implants. In certain preferred embodiments, the cells to be used are a fractionated population of immune cells. Recognized subpopulations of immune cells include the lymphocytes, such as B lymphocytes (Fc receptors, MHC classII, CD19+, CD21+), helper T lymphocytes (CD3+, CD4+, CD8−), cytolytic T lymphocytes (CD3+, CD4−, CD8+), natural killer cells(CD16+), the mononuclear phagocytes, including monocytes, neutrophils and macrophages, and dendritic cells. Other cell types that may be of interest include eosinophils and basophils.

Cells may be autologous (i.e., derived from the same individual) or syngeneic (i.e., derived from a genetically identical individual, such as a syngeneic littermate or an identical twin), although allogeneic cells (i.e., cells derived from a genetically different individual of the same species) are also contemplated. Although less preferred, xenogeneic (i.e., derived from a different species than the recipient) cells, such as cells from transgenic pigs, may also be administered. When the donor cells are xenogeneic, it is preferred that the cells are obtained from an individual of a species within the same order, more preferably the same superfamily or family (e.g., when the recipient is a human, it is preferred that the cells are derived from a primate, more preferably a member of the superfamily Hominoidea).

Cells may, where medically and ethically appropriate, be obtained from any stage of development of the donor individual, including prenatal (e.g., embryonic or fetal), infant (e.g., from birth to approximately three years of age in humans), child (e.g., from about three years of age to about 13 years of age in humans), adolescent (e.g., from about 13 years of age to about 18 years of age in humans), young adult (e.g., from about 18 years of age to about 35 years of age in humans), adult (from about 35 years of age to about 55 years of age in humans) or elderly (e.g., from about 55 years and beyond of age in humans).

In certain embodiments the cells to be labeled are stem cells. Stem cell therapies are commonly used as part of an ablative regimen for treatment of cancer with high dose radiation and/or chemotherapeutic agents. Ablative regimens generally employ hematopoietic stem cells, or populations of cells containing hematopoietic stem cells, as may be obtained, for example, from peripheral blood, umbilical cord blood or bone marrow. Cells of this type, or a portion thereof, may be labeled and tracked in vivo to monitor survival and engraftment at the appropriate location. Other types of stem cells are increasingly attractive as therapeutic agents for a wide variety of disorders.

As an example, cells may be mouse embryonic stem cells, or ES cells from another model animal. The labeling of such cells may be useful in tracking the fate of such cells administered to mice, optionally as part of a preclinical research program for developing embryonic stem cell therapeutics. Examples of mouse embryonic stem cells include: the JM1 ES cell line described in M. Qiu et al., Genes Dev 9, 2523 (1995), and the ROSA line described in G. Friedrich, P. Soriano, Genes Dev 5, 1513(1991), and mouse ES cells described in U.S. Pat. No. 6,190,910.

Many other mouse ES lines are available from Jackson Laboratories (Bar Harbor, Me.). Examples of human embryonic stem cells include those available through the following suppliers: Arcos Bioscience, Inc., Foster City, Calif., CyThera, Inc., San Diego, Calif., BresaGen, Inc., Athens, Ga., ES Cell International, Melbourne, Australia, Geron Corporation, Menlo Park, Calif., Goteborg University, Goteborg, Sweden, Karolinska Institute, Stockholm, Sweden, Maria Biotech Co. Ltd.-Maria Infertility Hospital Medical Institute, Seoul, Korea, MizMedi Hospital-Seoul National University, Seoul, Korea, National Centre for Biological Sciences/Tata Institute of Fundamental Research, Bangalore, India, Pochon CHA University, Seoul, Korea, Reliance Life Sciences, Mumbai, India, ReNeuron, Surrey, United Kingdom, StemCells, Inc., Palo Alto, Calif., Technion University, Haifa, Israel, University of California, San Francisco, Calif., and Wisconsin Alumni Research Foundation, Madison, Wis. In addition, examples of embryonic stem cells are described in the following U.S. patents and published patent applications: U.S. Pat. Nos. 6,245,566; 6,200,806; 6,090,622; 6,331,406; 6,090,622; 5,843,780; 20020045259; 20020068045. In preferred embodiments, the human ES cells are selected from the list of approved cell lines provided by the National Institutes of Health and accessible at http://escr.nih.gov. In certain preferred embodiments, an embryonic stem cell line is selected from the group consisting of: the WA09 line obtained from Dr. J. Thomson (Univ. of Wisconsin) and the UC01 and UC06 lines, both on the current NIH registry.

In certain embodiments, a stem cell for use in disclosed methods is a stem cell of neural or neuroendocrine origin, such as a stem cell from the central nervous system (see, for example U.S. Pat. Nos. 6,468,794; 6,040,180; 5,753,506; 5,766,948), neural crest (see, for example, U.S. Pat. Nos. 5,589,376; 5,824,489), the olfactory bulb or peripheral neural tissues (see, for example, Published US Patent Applications 20030003574; 20020123143; 20020016002 and Gritti et al. 2002 J Neurosci 22 (2): 437-45), the spinal cord (see, for example, U.S. Pat. Nos. 6,361,996, 5,851,832) or a neuroendocrine lineage, such as the adrenal gland, pituitary gland or certain portions of the gut (see, for example, U.S. Pat. No. 6,171,610 and PC12 cells as described in Kimura et al. 1994 J. Biol. Chem. 269: 18961-67). In preferred embodiments, a neural stem cell is obtained from a peripheral tissue or an easily healed tissue, thereby providing an autologous population of cells for transplant.

Hematopoietic or mesenchymal stem cells may be employed in certain disclosed methods. Recent studies suggest that marrow-derived hematopoietic (HSCs) and mesenchymal stem cells (MSCs), which are readily isolated, have a broader differentiation potential than previously recognized. Purified HSCs not only give rise to all cells in blood, but can also develop into cells normally derived from endoderm, like hepatocytes (Krause et al., 2001, Cell 105: 369-77; Lagasse et al., 2000 Nat Med 6: 1229-34). Similarly, HSCs from peripheral blood and from umbilical cord blood are expected to provide a useful spectrum of developmental potential. MSCs appear to be similarly multipotent, producing progeny that can, for example, express neural cell markers (Pittenger et al., 1999 Science 284:143-7; Zhao et al., 2002 Exp Neurol 174: 11-20). Examples of hematopoietic stem cells include those described in U.S. Pat. Nos. 4,714,680; 5,061,620; 5,437,994; 5,914, 108; 5,925,567; 5,763,197; 5,750,397; 5,716,827; 5,643,741; 5,061,620.

Examples of mesenchymal stem cells include those described in U.S. Pat. Nos. 5,486,359; 5,82,735; 5,942,225; 5,972,703, those described in PCT publication No:. WO 00/53795; WO 00/02654; WO 98/20907, and those described in Pittenger et al. and Zhao et al.

Stem cell lines are preferably derived from mammals, such as rodents (e.g. mouse or rat), primates (e.g. monkeys, chimpanzees or humans), pigs, and ruminants (e.g. cows, sheep and goats), and particularly from humans. In certain embodiments, stem cells are derived from an autologous source or an HLA-type matched source. For example, stem cells may be obtained from a subject in need of pancreatic hormone-producing cells (e.g. diabetic patients in need of insulin-producing cells) and cultured to generate autologous insulin-producing cells. Other sources of stem cells are easily obtained from a subject, such as stem cells from muscle tissue, stem cells from skin (dermis or epidermis) and stem cells from fat.

In some preferred embodiments, cells for administration to a human should be compliant with good tissue practice guidelines set by the U.S. Food and Drug Administration (FDA) or equivalent regulatory agency in another country. Methods to develop such a cell line may include donor testing, and avoidance of exposure to non-human cells and products.

Cells derived from a donor (optionally the patient is the donor) may be administered as unfractionated or fractionated cells, as dictated by the purpose of the cells to be delivered. Cells may be fractionated to enrich for certain cell types prior to administration. Methods of fractionation are well known in the art, and generally involve both positive selection (i.e., retention of cells based on a particular property) and negative selection (i.e., elimination of cells based on a particular property). As will be apparent to one of skill in the art, the particular properties (e.g., surface markers) that are used for positive and negative selection will depend on the desired population of cells. Methods used for selection/enrichment of cells may include immunoaffinity technology or density centrifugation methods. Immunoaffinity technology may take a variety of forms, as is well known in the art, but generally utilizes an antibody or antibody derivative in combination with some type of segregation technology. The segregation technology generally results in physical segregation of cells bound by the antibody and cells not bound by the antibody, although in some instances the segregation technology which kills the cells bound by the antibody may be used for negative selection.

Any suitable immunoaffinity technology may be utilized for selection/enrichment of the selected cells to be used, including fluorescence-activated cell sorting (FACS), panning, immunomagnetic separation, immunoaffinity chromatography, antibody-mediated complement fixation, immunotoxin, density gradient segregation, and the like. After processing in the immunoaffinity process, the desired cells (the cells bound by the immunoaffinity agent in the case of positive selection, and cells not bound by the immunoaffinity agent in the case of negative selection) are collected and either subjected to further rounds of immunoaffinity selection/enrichment, or reserved for administration to the patient.

Immunoaffinity selection/enrichment is typically carried out by incubating a preparation of cells comprising the desired cell type with an antibody or antibody-derived affinity agent (e.g., an antibody specific for a given surface marker), then utilizing the bound affinity agent to select either for or against the cells to which the antibody is bound. The selection process generally involves a physical separation, such as can be accomplished by directing droplets containing single cells into different containers depending on the presence or absence of bound affinity agent (FACS), by utilizing an antibody bound (directly or indirectly) to a solid phase substrate (panning, immunoaffinity chromatography), or by utilizing a magnetic field to collect the cells which are bound to magnetic particles via the affinity agent (immunomagnetic separation). Alternately, undesirable cells may be eliminated from the preparation using an affinity agent which directs a cytotoxic insult to the cells bound by the affinity agent. The cytotoxic insult may be activated by the affinity agent (e.g., complement fixation), or may be localized to the target cells by the affinity agent (e.g., immunotoxin, such as ricin B chain).

6. Methods for Labeling Cells

A variety of methods may be used to label cells with imaging or proxy reagents. In general, cells will be placed in contact with imaging reagent such that the imaging reagent becomes associated with the cell. Conditions will often be standard cell culture conditions designed to maintain cell viability. The term “associated” is intended to encompass any manner by which the imaging reagent and cell remain in sufficiently close physical proximity for a sufficient amount of time as to allow the imaging reagent to provide useful information about the position of the cell, whether in vivo or in vitro. Imaging reagent may be located intracellularly, e.g. after phagocytosis, fluid-phase endocytosis, receptor mediated endocytosis, addition of cationic entities, transfection, electroporation, or surfactant mediated entry into the cell. Immune cells, such as dendritic cells, macrophages and T cells are highly phagocytic and data presented herein and in other studies demonstrate that such cells, and other phagocytic cell types, are readily labeled.

Imaging reagent may be inserted into a cell membrane or covalently or non-covalently bound to an extracellular component of the cell. For example, certain linear fluorocarbons described herein may be derivatized to attach one or more targeting moiety. A targeting moiety will be selected to facilitate association of the imaging reagent with the cell to be labeled. A targeting moiety may be designed to cause non-specific insertion of the fluorocarbon into a cell membrane (e.g., a hydrophobic amino acid sequence or other hydrophobic moiety such as a palmitoyl moiety or myristoyl moiety) or to facilitate non-specific entry into the cell. A targeting moiety may bind to a cell surface component, as in the case of receptor ligands. A targeting moiety may be a member of a specific binding pair, where the partner is a cell surface component. The targeting moiety may be, for example, a ligand for a receptor, or an antibody, such as a monoclonal or polyclonal antibody or any of the various polypeptide binding agents comprising a variable portion of an immunoglobulin (e.g., Fv fragment, single chain Fv(scFv) fragment, Fab′ fragment, F (ab′) 2 fragment, single domain antibody, camelized antibody, humanized antibody, diabodies, tribodies, tetrabodies).

In many embodiments, cells are labeled by contacting the cells with an emulsion of the imaging reagent, such that the agent is taken up by cells. Both phagocytic and non-phagocytic cells may be labeled by such a method.

For labeling cells, the imaging reagents can be employed in one or more of at least three modalities: 1) imaging reagents that are internalized or otherwise absorbed by target cells without the formation of any covalent or other binding association; 2) imaging reagents that covalently attach to target cells; and 3) imaging reagents coupled to molecules, such as antibodies or ligands, that bind to molecules present on the target cells.

Imaging reagents of the first type include the perfluoro crown ethers and other PFPEs that are taken up by cells and, preferably, are retained in the cell without degradation for a substantial period of time, e.g., having a half-life in the cell of at least 1 hour, at least 4 hours, at least about a day, at least about three days, or even at least about a week. For obvious reasons, it is preferred that the imaging reagent not interfere with ordinary cellular functions or exhibit cytotoxicity at the concentrations employed for labeling. As demonstrated herein, perfluoropolyethers show minimal toxic effect on the labeled cells.

Imaging reagents of the second type include electrophilic compounds that react with nucleophilic sites on the cell surface, such as exposed thiol, amino, and/or hydroxyl groups. Accordingly, imaging reagents such as maleimides, alkyl iodides, N-hydroxysuccinimide or N-hydroxysulfosuccinimide esters (NHS or sulfo-NHS esters), acyl succinimides, and the like can form covalent bonds with cell surfaces.

Other techniques used in protein coupling can be adapted for coupling imaging reagents to cell surface proteins. See Means et al. (1990) Bioconjugate Chemistry 1: 2-12, for additional approaches to such coupling.

Imaging reagents of the third type can be prepared by reacting imaging reagents of the second type not with the cells themselves, but with a functional moiety that is a cell-targeting ligand or antibody. Suitable ligands and antibodies can be selected for the application of interest. For example, a ligand that selectively targets hematopoietic cells could be labeled with an imaging reagent as described herein and administered to a patient, such as by injection.

Alternatively, an imaging reagent can be coupled to an indiscriminate internalizing peptide, such as antennapedia protein, HIV transactivating (TAT) protein, mastoparan, melittin, bombolittin, delta hemolysin, pardaxin, Pseudomonas exotoxin A, clathrin, Diphtheria toxin, C9 complement protein, or a fragment of any of these. Cells treated with this indiscriminate molecule ex vivo will absorb the imaging reagent. When such labeled cells are implanted into an animal, such as a mammal, the imaging reagent can be used to visualize and/or track the implanted cells by in vivo imaging techniques.

In one embodiment, the internalizing peptide is derived from the drosophila antennapedia protein, or homologs thereof. The 60-amino acid-long homeodomain of the homeo-protein antennapedia has been demonstrated to translocate through biological membranes and can facilitate the translocation of heterologous polypeptides to which it is coupled. See for example Derossi et al. (1994) J Biol Chem 269: 10444-10450; and Perez et al. (1992) J Cell Sci 102: 717-722. It has been demonstrated that fragments as small as 16 amino acids long of this protein are sufficient to drive internalization. See Derossi et al. (1996) J Biol Chem 271: 18188-18193.

Another example of an internalizing peptide is the HIV transactivator (TAT) protein. This protein appears to be divided into four domains (Kuppuswamy et al. (1989) Nucl. Acids Res. 17: 3551-3561). Purified TAT protein is taken up by cells in tissue culture (Frankel and Pabo, (1989) Cell 55: 1189-1193), and peptides, such as the fragment corresponding to residues 37-62 of TAT, are rapidly taken up by celli71 vitro (Green and Loewenstein, (1989) Cell 55: 1179-1188). The highly basic region mediates internalization and targeting of the internalizing moiety to the nucleus (Ruben et al., (1989) J. Virol. 63: 1-8). Peptides or analogs that include a sequence present in the highly basic region can be conjugated to fluorinated imaging reagents to aid in internalization and targeting those agents to the intracellular milieu.

In some embodiments, imaging reagents are introduced into host cells via liposomes. A liposome is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Cationic liposomes, made with cationic lipids such as DOTMA, DOSPA, and DMRIE, form complexes with DNA. These complexes bind to the surface of cells and internalize to endosomes. The preparation and use of liposome complexes for delivery of expression vectors into eukaryotic cells is well known, and lipids for liposome preparation are commercially available (for example, from Invitrogen Corp., Carlsbad, Calif.). Administering liposome complexes to cells generally involves contacting target cells with the complex.

In some embodiments cell targeting of imaging reagents is accomplished by a generic receptor-targeted polymer nanocontainer platform (Broz et al., 2005, J Control Release. February 2; 102(2):475-88.

7. High Throughput Techniques

a. Methods for Determining Optimal Labeling Parameters

Using standard sterile tissue culture practices one can associate the labeling agent with the cell of interest. In some embodiments the cells of interest are a plurality of sub-samples comprising cells taken from a cell sample. In some embodiments a sample is from a subject such as a human patient.

The methods of this invention rely on the use of multi-well tissue culture plates (e.g., a polystyrene plate, a polypropylene plate, a polycarbonate plate, etc., such as those produced by Corning, or equivalently multiple culture vials or tubes) in order to conduct multiple labeling experiments under different conditions simultaneously. Multi-well tissue culture plates can contain, for example, 2, 4, 6, 8, 12, 24, 48, 96, 384, 1536 individual wells, each containing a test article. In preferred embodiments, each well will have the same number of cells of interest (i.e., test article) in culture medium. For example, a single multi-well plate will contain test articles of a particular cell type, or the cells from a particular patient who will ultimately undergo a cellular therapeutic procedure. Optionally, each culture well will contain different numbers of cells, cells of different types, or cells from different patients. The culture medium may be comprised of: bulk ions, trace elements, sugars, amino acids, vitamins, choline, inositol, serum, peptide hormones or hormone-like growth factors, and antibiotics.

The labeling agent is then added to each well containing cells. This step is performed immediately after the cells are deposited into the wells. Optionally the labeling agent can be added at various times (e.g., 15 minutes, 1 hour, 4 hours, 12 hours, 24 hours, etc.) after the cells are “plated” in the wells, perhaps to allow for cellular adherence to the well bottom. Optionally during this wait time the multi-plate well can be stored in a tissue culture incubator at near physiological temperatures (described below). In preferred embodiments the labeling agent concentration in the culture medium is varied in different wells in a systematic manner within a given plate. For example the agent concentration can be incremented across rows or columns, where a given row or column will have the same concentration. This concentration redundancy among the wells in a given row or column can be used to generate the mean behavior of the cells contacted with a particular agent concentration, as well as a measure of the statistical variance. Concentration redundancy among wells can also be achieved by many other different arrangements (e.g., clustering, periodic lattices, random). Optionally, each individual well has a different, pre-determined agent concentration.

Normally, after the cells and agent have been added to the wells, the plate will be placed in a tissue culture incubator. Often tissue culture incubators maintain physiological environments for the cells; standard culture conditions are 37° C. in a humidified 5% CO2 and 95% air atmosphere, however different temperatures and atmospheric conditions can be used, depending on the cell type.

Control over the incubation environment can be used to modulate the label uptake, for example by controlling temperature time course (e.g., going from 37 to 4 deg), or by adding additional substances to the culture medium that can modulate endocytosis or other uptake mechanisms (e.g., Faria et al., 2006, FEBS lett. January 9; 580(1): 155-60; Kreuser et al., 1995, Recent Results Cancer Res.; 139:371-82). Tissue culture methods are well established in the art, [Helgason et al., Basic Cell Culture Protocols (Methods in Molecular Biology)].

b. Detection Methods

In certain embodiments the disclosure uses high-throughput instruments for detecting the amount of labeling agent that becomes associated with the cellular test articles. Above, various types of proxy reagents that can be associated with the cells were described. The amount of agent detected in a well may correlate with the degree of agent loading for that particular condition. In preferred embodiments of the invention one uses quantitative instruments for the purpose of assaying the amount of agent per well or for sample tubes. Furthermore, for rapidity, economy, and/or accuracy it is preferred that these measurements are made in an automated or semi-automated fashion. Depending on the class of the analogue or dual mode agents used (described above) there are many types of commercially-available instruments that can be used for this purpose that can be used in conjunction with multi-well plates or multiple sample tubes spectrophotometers for use with optically absorbent agents, fluorescent dyes, genetically encoded fluorescent agents, luminescent agents and luciferases or colorimetric agents (e.g., Hitachi, F-4500 and Beckman Coulter, BR-9741B). Scintillation counters could be used with radioactive isotopes (e.g., Beckman Coulter, Wallac, and Perkin Elmer)

Additional examples of devices that can be used to read-out the agent labeling information are high-throughput or high-content automated microscope systems (U.S. Pat. No. 6,775,567 and US patent application 20030103662), such as those developed by Cellomics Inc. (ArrayScan®, KineticScan® and cellWoRx) or equivalent devices, high-throughput and/or automated fluorescent activated cell sorting (FACS) devices (e.g., Becton Dickson and Beckman Coulter), and high-throughput, microliter scaled, cell analysis systems (Guava Technologies, Inc., Hayward, Calif.)

8. Additional Assays

In addition to assessing imaging reagent uptake in cells, the same multi-well or multi sample preparations can be used to assay other biological characteristics and changes to the cell as a consequence of the labeling procedures. Following labeling, characteristics such as total cell number, MTT, total double-stranded DNA, tripan-blue exclusion, high-throughput automated microscopy, immunostaining assays, etc. Auxiliary assays such as these can be used to confirm, for example, that the labeling process was not overtly toxic or in someway harmful to the cell. These assays can be performed in situ on the same plates or tubes used to read-out the dose of MRI/MRS label. For example, one can perform cell counts in the wells after labeling, which may indicate the amount of cell death, using automated microscopes that can perform autonomous cell counting within each well of a multi-well plate.

Additional examples of complementary assays that can be performed to assess the impact of labeling on cells comprise: cell cycle assays (e.g. DNA content analysis, BrdU incorporation assays or cell cycle marker detection), migration assays, and cell type-specific functional assays.

Those skilled in the art can imagine numerous other types of in vitro assays that provide relevant biological information that can be performed on the labeled cells while in the multi-well plates. Assays can also include those that are part of the routine protocol for preparing the cells for therapeutic delivery to the patient, which are unrelated to the labeling process or associated manipulations.

### EXEMPLIFICATION

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples.

### Example 1

### Analysis of the Use of Proxy Agents

As an example of the feasibility of these methods, we will analyze whether fluorescent-MRI dual-mode agents can be synthesized, can be used to label cells with similar characteristics as the non-fluorescent versions, and whether there is correlation between fluorescence intensity and the 19F content of the cell. Thus, tissue culture protocols that are developed for labeling cells can be used for both fluorescent and non-fluorescent versions of the PFPE nanoparticles. Furthermore, fluorescent analogs or dual mode agents can be used for in vitro measurements of the key parameter Fc as part of the cell labeling protocol development and/or validation. For example, the mean cell loading, measured by Fc, could be evaluated using low-cost fluorimeters, rather than expensive 19F NMR instrumentation, and the Fc result could be used for subsequent in vivo experiments using the non-fluorescent versions of the PFPE.

Linear PFPE molecule is conjugated to commercially-available dyes such as BODIPY-TR dye and Alexa 647. Emulsions are made using blended mixtures of the PFPE-dye conjugate and the non-conjugated PFPE. It is expected that the materials show a small particle size of comparable size as the non-fluorescent versions, the fluorescent properties are maintained, the materials show a dose dependent fluorescence intensity, the fluorescent spectrum is unaffected by the conjugation to the PFPE, and labeled cells exhibit similar cell loading characteristics as the non-fluorescent equivalents.

It is expected that cells labeled with the dual mode agent will show a linear correlation with 19F NMR-measured uptake. Thus, with suitable fluorescent calibration standards, one can measure the PFPE loading and the cellular dose of the reagents (i.e., Fc) on the basis of the fluorescent measurement alone. This measurement can then be used to achieve consistent loading of cells of interest, and the Fc parameter is used to aid in cell quantification in vivo using 19F MRI/MRS as described above.

### Example 2

### Assessment of Cell Labeling with a Fluorescent Proxy Agent

Dual mode agents or other proxy agents that show a linear correlation with 19F NMR-measured uptake will be used to assess cell labeling for previously untested cell types and patient samples in high-throughput format according to the methods of the application.

Cells are plated in a 384-well plate format, and the agent concentration to be added to the culture medium is varied in order to identify the most effective labeling dose that does not produce toxicity. The parameters for labeling known cells will be used as a starting point. The fluorescent labeling of cells is analyzed using a high-throughput automated microscope. Other assays to examine toxicity of the labeling reagent such as tripan-blue exclusion are also conduced. Control experiments using an in vivo imaging reagent analyzed by MRI are also conduced.

It is expected that the proxy agent and in vivo MRI agent will reveal equivalent dosing information in the cells. Based on the results, an effective dose for the new cell type or patient sample will be chosen for future in vivo experiments.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method for assessing the labeling of cells intended for administration to a patient, the method comprising:
a) contacting cells of a plurality of substantially identical cell samples with an in vivo imaging reagent;
b) detecting the in vivo imaging reagent in the samples; and
c) assessing the labeling of the cells with the in vivo imaging reagent.

2. A method for assessing the labeling of cells intended for administration to a patient, the method comprising:
a) contacting cells of a plurality of substantially identical cell samples with an in vivo imaging reagent and a proxy reagent;
b) detecting the proxy reagent in the samples; and
c) assessing the labeling of the cells with the proxy reagent, thereby assessing the labeling of the cells with the in vivo imaging reagent.

3. The method of claim 1, wherein at least two of the substantially identical cell samples are subjected to predetermined conditions that differ from each other, and wherein the method further comprises assessing the effects of differing conditions on the labeling of the cells with the in vivo imaging reagent.

4. The method of claim 3, wherein at least ten substantially identical cell samples are subjected to predetermined conditions that differ from each other.

5. The method of claim 4, wherein the cells are contacted with the in vivo imaging reagent or the in vivo imaging reagent and the proxy reagent by use of automated, high-throughput technology.

6. The method of claim 5, wherein automated, high-throughput technology is used to assess the labeling of the cells with the in vivo imaging reagent.

7. The method of claim 1, wherein the in vivo imaging reagent is an agent that is suitable for detection in vivo by a nuclear magnetic resonance technique.

8. The method of claim 7, wherein in vivo imaging reagent is selected from the group consisting of: paramagnetic imaging agents, superparamagnetic iron-oxide particles, magnetite particles, fluorocarbon imaging reagents, Gd chelates, and Mn chelates.

9. The method of claim 8, wherein the in vivo imaging reagent comprises PFPE emulsions.

10. The method of claim 1, wherein the in vivo imaging reagent is an agent that is suitable for detection by a non-invasive imaging technique selected from the group consisting of: positron emission tomography (PET), gamma ray detection, or SPECT (single-photon emission computed tomography).

11. The method of claim 2, wherein the proxy reagent is selected from the group consisting of: a fluorescent or luminescent protein, a fluorescent or luminescent analogue agent, a fluorescent or luminescent dye, a colorimetric agent, or a radioactive agent.

12. The method of claim 2, wherein the in vivo imaging reagent is stably associated with the proxy reagent to form a dual imaging reagent.

13. The method of claim 12, wherein the in vivo imaging reagent is an agent that is suitable for detection in vivo by a nuclear magnetic resonance technique.

14. The method of claim 12, wherein the proxy reagent is selected from the group consisting of: a fluorescent or luminescent protein, a fluorescent or luminescent analogue agent, a fluorescent or luminescent dye, a colorimetric agent, or a radioactive agent.

15. The method of claims 3, wherein the predetermined conditions that differ from each other differ in one or more aspect selected from the group consisting of: temperature, atmospheric composition, humidity, culture medium concentration of the in vivo labeling agent, duration of exposure to the in vivo labeling agent, concentration of an uptake enhancing agent and duration of exposure to an uptake enhancing agent.

16. The method of claim 1, further comprising determining conditions for labeling the cells with the in vivo imaging reagent that will allow the preparation of a dosage of the cells for administration to a patient, wherein the dosage conforms to known safety parameters associated with the in vivo imaging reagent and provides adequate labeling to permit detection of the labeled cells in vivo.

17. The method of claim 1, further comprising administering to the patient a dosage of the cells, wherein the dosage conforms to known safety parameters associated with the in vivo imaging reagent and provides adequate labeling to permit detection of the labeled cells in vivo.

18. The method of claim 3, further comprising an additional assay selected from the group consisting of: a viability assay, a cell count, a cell cycle assay, a migration assay, and a functional assay.

19. The method of claim 1, wherein the patient is a mammal.

20. The method of claim 1, wherein the patient is a human.

21. The method of claim 1, wherein the cells are autologous to the patient.

22. The method of claim 1, wherein the cells are allogeneic to the patient.

23. The method of claim 1, wherein cells are selected from the group comprising: blood cells, myoblasts, bone marrow cells, peripheral blood cells, umbilical cord blood cells, cardiomyocytes, chondrocytes, immune cells, fetal neural cells, neuronal precursors, fibroblasts, hepatocytes, islet cells of pancreas, keratinocytes and precursors of any of the preceding.

24. The method of claim 1, wherein the cells are selected from the group consisting of: embryonic stem cells, cells cultured from embryonic stem cells, adult stem cells and cells cultured from adult stem cells.

25. The method of claim 1, wherein the substantially identical samples are each a portion of a sample of cells obtained from a donor.

26. The method of claim 1, wherein the substantially identical samples are each a portion of cells cultured from cells derived from a donor.

27. A composition for labeling cells, the composition comprising an in vivo imaging reagent in stable association with a protein proxy reagent.

28. The composition of claim 27, wherein the protein proxy reagent is selected from the group consisting of: a fluorescent protein, a luminescent protein, a colored protein, a fluorogenic protein, a luminogenic protein and a chromogenic protein.

29. The method of claim 1, wherein assessing the labeling of the cells with the in vivo imaging reagent comprises quantifying the amount of in vivo imaging reagent that is associated with the cells.

30. The method of claim 2, wherein assessing the labeling of the cells with the in vivo imaging reagent comprises quantifying the amount of proxy reagent that is associated with the cells.

31. A method of administering a safe and useful dosage of labeled cells to a patient and detecting said labeled cells in vivo, the method comprising:
a) contacting cells of a plurality of substantially identical cell samples with an in vivo imaging reagent;
b) detecting the in vivo imaging reagent in the samples;
c) assessing the labeling of the cells with the in vivo imaging reagent;
d) determining conditions for labeling the cells with the in vivo imaging reagent that will allow the preparation of a dosage of the cells for administration to a patient, wherein the dosage conforms to known safety parameters associated with the in vivo labeling agent and provides adequate labeling to permit detection of the labeled cells in vivo;
e) administering the dosage of the cells to the patient; and
f) detecting the labeled cells in vivo by a non-invasive imaging technique.

32. The method of claim 31, wherein the non-invasive imaging technique in a nuclear magnetic resonance technique.

33. A method of administering a safe and useful dosage of labeled cells to a patient and detecting said labeled cells in vivo, the method comprising:
a) contacting cells of a plurality of substantially identical cell samples with an in vivo imaging reagent and a proxy reagent;
b) detecting the proxy reagent in the samples;
c) assessing the labeling of the cells with the proxy reagent, thereby assessing the labeling of the cells with the in vivo imaging reagent;
d) determining conditions for labeling the cells with the in vivo imaging reagent that will allow the preparation of a dosage of the cells for administration to a patient, wherein the dosage conforms to known safety parameters associated with the in vivo labeling agent and provides adequate labeling to permit detection of the labeled cells in vivo;
e) administering the dosage of the cells to the patient; and
f) detecting the labeled cells in vivo by a non-invasive imaging technique.

34. The method of claim 33, wherein the non-invasive imaging technique in a nuclear magnetic resonance technique.
